(19) United States
(12) Patent Application Publication
(10) Pub. No.: US 2021/0243878 A1
(43) Pub. Date: Aug. 5, 2021

(54) A VARIABLE-ENERGY PROTON LINEAR ACCELERATOR SYSTEM AND A METHOD OF OPERATING A PROTON BEAM SUITABLE FOR IRRADIATING TISSUE

(71) Applicant: ADAM S.A., Meyrin (CH)

(72) Inventor: Giovanni DE MICHELE, Eindhoven (NL)

(73) Assignee: ADAM S.A., Meyrin (CH)

(21) Appl. No.: 17/049,773

(22) PCT Filed: Apr. 24, 2019
(86) PCT No.: PCT/EP2019/060469

§ 371 Date: Oct. 22, 2020

(30) Foreign Application Priority Data
Apr. 25, 2018 (EP) 18169362.3

(51) Int. Cl. H05H 9/04; H05H 7/00; A61N 5/10; H05H 7/02

(52) U.S. Cl. CPC H05H 9/047; H05H 7/001; A61N 5/1067; H05H 2007/004; A61N 2005/1087; H05H 2007/022; H05H 7/02

(57) ABSTRACT

One of the obstacles to the widespread use of proton therapy is the availability of affordable and compact proton sources and accelerators. The use of linear accelerators allow the construction of such a compact source which may be installed in existing medical facilities. However, instability occurs after accelerating units are turned on or off. A proton linear accelerator system configured to provide RF energy during the off-time of the proton beam operating cycle may be used for increasing or maintaining the temperature of cavities. A method of operating a proton beam is also provided which is suitable for irradiating tissue. These may provide an improved settling time.

### FIELD OF THE INVENTION

The invention relates to a proton linear accelerator system for irradiating tissue comprising a proton source for providing a proton beam during operation.

### BACKGROUND OF THE INVENTION

Energetic beams, such as X-rays, have been used therapeutically for many years to damage the DNA of cancer cells and to kill them in humans and animals. However, during the treatment of tumors, the X-rays expose surrounding healthy tissues, particularly along the path of the X-rays through the body, both before (entrance dose) and after (exit dose) the tumor site. The X-ray dose is frequently sufficiently high to result in short-term side effects and may result in late carcinogenesis, growth dysfunction in the healthy tissue and growth retardation in the case of children.

Proton beams are a promising alternative because they may also destroy cancer cells, but with a greatly reduced damage to healthy tissue. The energy dose in tissue may be concentrated at the tumor site by configuring the beam to position the Bragg Peak proximate the tumor, greatly reducing the dose on the entrance treatment path, and in many cases almost completely eliminating the exit dose on the treatment path. The longitudinal range of a proton beam in tissue is generally dependent upon the energy of the beam. Here dose is used to indicate the degree of interaction between the beam and tissue—interaction is minimal until the end portion of the beam range, where the proton energy is deposited in a relatively short distance along the beam path. This reduction in unwanted exposure longitudinally before and after the target site means that improved doses may be delivered without compromising surrounding healthy tissue. This may reduce the length of treatment, by allowing the delivery of a higher differential effective dose to the tumor itself, above and beyond the dose which is absorbed before and after the tumor, and typically reduces side-effects due to the correspondingly lower surrounding dose. It is particularly beneficial when treating tumors located near critical organs or structures such as the brain, the heart, the prostate or the spinal cord, and when treating tumors in children. Its accuracy makes it also particularly effective when treating ocular tumors. In addition, proton beams may be accurately positioned and deflected to provide transverse control of beam paths.

One of the obstacles to the widespread use of proton therapy is the availability of affordable and compact proton sources and accelerators. The energy of the protons used for treatment are usually in the range 50-300 MeV, and more typically in the range 70-250 MeV. Existing sources relying on cyclotrons or synchrotrons are very large, require custom-built facilities, and are expensive to build and maintain. The use of linear accelerators (Linacs) allow the construction of such a compact source which may be installed in existing medical facilities.

The longitudinal position (depth) of the proton energy dose is mainly configured by changing the energies of the protons (usually measured in MeV) in the beam. U.S. patent Ser. No. 05/382,914 describes a compact proton-beam therapy linac system utilizing three stages to accelerate the protons from the proton source: a radio-frequency quadrupole (RFQ) linac, a drift-tube linac (DTL) and a side-coupled linac (SCL). The SCL comprises up to ten accelerator units arranged in a cascade, each unit being provided with an RF energy source. The treatment beam energy is controlled by a coarse/fine selection system—in the coarse adjustment, turning one or more of the accelerator units off provides eleven controlled steps from 70 MeV to 250 MeV, with each step being approximately 18 MeV. Fine adjustment of the beam energy between these steps is performed by inserting degrading absorbers, such as foils, into the beam.

The disadvantage of such a system is that after each switching step, the proton-beam system requires some time for the beam energy to stabilize before it may be used for therapy. In addition, the actuation systems for the degrading foils are often unreliable, and the foils must be regularly replaced.

From PCT application WO 2018/043709 A1, it is known to introduce a random component into the generation moment of the proton beam pulses, which are subsequently accelerated for use in semiconductor manufacturing. This is done to reduce the noise which may accumulate inside a high frequency cavity, due to the excitation of higher order modes which may generate heat. Providing slightly different frequency shifts may reduce resonant amplification, and may therefore also reduce the heating of the cavity.

From PCT application WO 2015/175751 A1, it is known to inject two different electron beam current amplitudes within the same RF pulse to produce two endpoint energies of accelerated electrons for producing X-rays for cargo inspection.

### OBJECT OF THE INVENTION

It is an object of the invention to provide a proton linear accelerator system for irradiating tissue with an improved beam energy control.

### SUMMARY OF THE INVENTION

A first aspect of the invention provides a proton linear accelerator system for irradiating tissue, the accelerator system comprising: a proton source for providing a proton beam during operation; a beam output controller for adjusting the beam current of the proton beam exiting the source; a first accelerator unit having: a first proton beam input for receiving the proton beam; a first proton beam output for exiting the proton beam; a first RF energy source for providing RF energy during operation; at least one first cavity extending from the first proton beam input to the first proton beam output, for receiving RF energy from the first energy source and for coupling the RF energy to the proton beam as it passes from the first beam input to the first beam output; the system further comprising: an RF energy controller connected to the first RF energy source for adjusting the RF energy provided to the at least one first cavity and further connected to the beam output controller; the beam output controller being configured to provide proton beam pulses with a predetermined and/or controlled beam operating cycle; and the RF energy controller being configured to provide RF energy during the off-time of the proton beam operating cycle such that the temperature of the first cavity is increased or maintained.

The invention is based upon the insight that applying substantially constant RF power to the accelerator units that are inactive (providing little, negligible or zero acceleration) or partially active (providing some acceleration) for a given output energy allows a very quick recovery when they are needed to increase the energy of the beam. The RF energy provided may be predetermined and/or controlled to increase or maintain the temperature of the cavity.

During operation of the system for proton therapy, the damage to surrounding tissue may be reduced by changing the beam energy, and therefore both the range of the beam and the corresponding Bragg peak. By adjusting the depth of the Bragg peak many separate Bragg peaks may be overlapped to produce an extended Bragg peak which produces a flat, or approximately flat, dose distribution which covers the tumor region. It is therefore advantageous to have a relatively small time between energy steps as this reduces the total treatment time, thereby reducing the risk of patient movement during treatment. Additionally or alternatively, the number of energy levels available for treatment may be increased, allowing a more accurate control of the spread of energy to surrounding tissues. Additionally or alternatively, movements of the tumor during treatment due to, for example patient breathing, may also be compensated for in real-time to improve the control even further.

A further aspect of the invention provides an accelerator system wherein the RF energy controller is further configured to provide substantially the same RF energy for each successive proton beam operating cycle.

This provides a high degree of stability to the accelerator system by providing an improved settling-time after beam energy change. In some embodiments, the settling-time may be substantially negligible.

Another aspect of the invention provides an accelerator system where the RF energy controller is further configured to provide RF energy during both the on-time and the off-time of the proton beam operating cycle.

This provides a high degree of stability to the accelerator system by providing an improved settling time when a treatment beam is being provided—the RF energy during the on-time transfers energy to the proton beam, and the RF energy during the off-time increases or maintains the temperature of the cavity.

Yet another aspect of the invention provides an accelerator system further comprising: a second accelerator unit having: a second proton beam input for receiving the proton beam from the first accelerator unit; a second proton beam output for exiting the proton beam; a second RF energy source for providing RF energy during operation; at least one second cavity extending from the second proton beam input to the second proton beam output, for receiving RF energy from the second energy source and for coupling the RF energy to the proton beam as it passes from the second beam input to the beam output; the RF energy controller being further connected to the second RF energy source for adjusting the RF energy provided to the at least one second cavity; and the RF energy controller being configured to provide RF energy during the off-time of the proton beam operating cycle such that the temperature of the second cavity is increased or maintained.

A plurality of accelerator units may be cascaded to provide a stepwise increase in the energy of the proton beam. Each accelerator unit may be operated to increase the energy of the proton beam by a fixed or variable amount.

The accelerator system may optionally be configured to provide RF energy to the first and second cavities which is substantially the same.

By configuring the energy increase of the proton beam by each accelerator (from a plurality of accelerator units) to be substantially identical, the number of proton beam energy settings will be related to the number of accelerating units in the cascade.

In yet another aspect of the invention, a method of operating a proton beam is provided which is suitable for irradiating tissue, the method comprising: providing proton beam pulses with a predetermined and/or controlled beam operating cycle from a proton beam source; adjusting the beam current of the proton beam exiting the source; providing RF energy from a first RF energy source to at least one first cavity; coupling the RF energy to the proton beam as it passes through the at least one cavity; and adjusting the RF energy provided to the at least one first cavity to provide RF energy during the off-time of the proton beam operating cycle such that the temperature of the first cavity is increased or maintained.

Optionally, the RF energy may be adjusted to provide substantially the same RF energy for each successive proton beam operating cycle. Additionally or alternatively, the RF energy may also be adjusted to provide RF energy during both the on-time and the off-time of the proton beam operating cycle.

These and other aspects of the invention are apparent from and will be elucidated with reference to the embodiments described hereinafter.

### BRIEF DESCRIPTION OF THE DRAWINGS

In the drawings:

FIG. 1 schematically shows a proton linear accelerator system according to the invention,

FIG. 2 schematically depicts an accelerating stage comprising one or more cascaded accelerator units,

FIG. 3 schematically depicts a first and second cascaded accelerator unit,

FIGS. 4A and 4B depict two possible variations in beam energy with the RF energy pulse required to provide a substantially constant average RF power,

FIGS. 4C and 4D depict two possible examples of operation of an accelerating unit in an improved non-accelerating mode,

FIG. 5A depicts an RF drive envelope for approximately 50% energy gain with a substantially constant RF energy per pulse,

FIG. 5B depicts the calculated accelerator field response envelope for the RF drive envelope depicted in FIG. 5A,

FIG. 6A depicts schematically a block diagram of a suitable low-level RF unit employing a DDS chip,

FIG. 6B shows the phasor diagram of the two signals used to modulate the amplitude and phase of the RF drive envelope made of two adjacent pulses,

FIG. 7A depicts beam control configurations that keep the average power substantially constant by alternating pulses with and without the proton beam, and

FIG. 7B depicts beam control configurations that keep the average power substantially constant by dividing each pulse into two intervals, one with proton beam and one without.

### DETAILED DESCRIPTION OF THE INVENTION

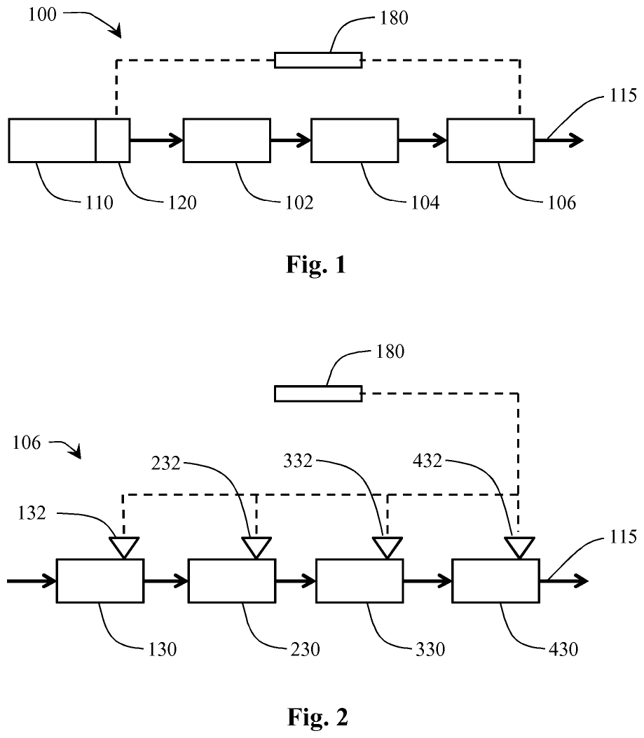

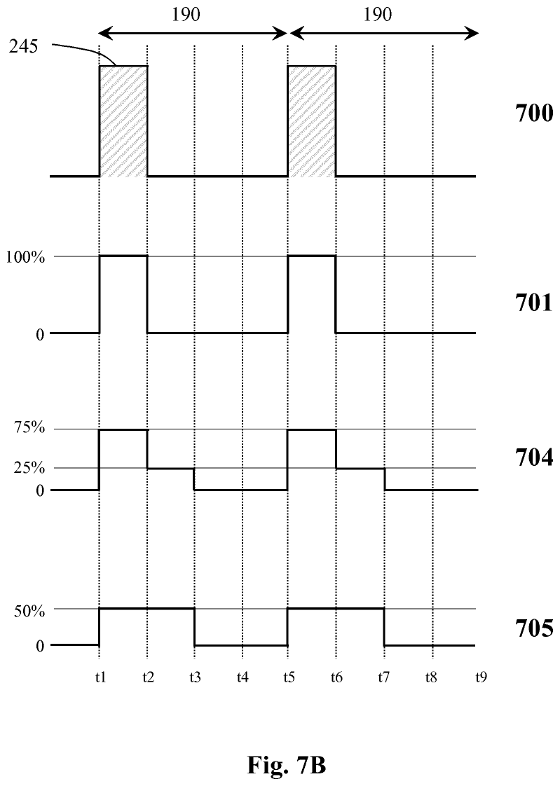

FIG. 1 schematically shows a proton linear accelerator (or linac) system 100 according to the invention. The linac system 100 comprises a proton beam source 110 for providing a proton beam 115 during operation. A beam output controller 120 is provided to adjust the beam current of the proton beam exiting the source 110. The proton beam 115 exiting the beam controller 120 is a pulsed beam. It may also be advantageous to configure the beam controller 120 to vary the proton beam duty cycle 145, 245. The beam output controller 120 may also be configured to blank the beam for one or more proton beam duty cycles 190. As depicted in FIGS. 7A and 7B, the operating cycle 190 of the proton beam 115 comprises an on-time and an off-time—the on-time is when the proton beam 115 energy is greater than zero, and the off-time is when the proton beam 115 energy is substantially lower than the on-time energy. The proton beam duty cycle 145, 245 is the on-time expressed as a fraction of the operating cycle 190 period, and often specified as a percentage or ratio. Typically, the energy during the off-time is less than or equal to the minimum energy required for operation of the proton accelerator system 100. The energy during the on-time is usually sufficient for therapeutic purposes and may contribute to the therapeutic dose delivered to the patient.

One or more accelerating stages 102,104,106 are provided to increase the beam energy to levels typically required for therapy of 50-300 MeV, and more typically in the range 70-250 MeV. Any suitable acceleration techniques may be used that are known to the skilled person.

The proton beam 115 exiting the beam controller 120 enters the first accelerating stage 102. In this particular embodiment, the first stage 102 may be provided by an RFQ (Radio-Frequency Quadrupole) which accelerates the beam up to approximately 3 to 10 MeV, preferably 5 MeV. In a first example, a suitable RFQ 102 may operate at a frequency of 750 MHz, with a vane-to-vane voltage of 68 kV, a beam transmission of 30% and a required RF power of 0.4 MW. In a second example, a suitable RFQ 102 may operate at a frequency of 499.5 MHz, with a vane-to-vane voltage of 50 kV, a beam transmission of 96% and a required RF power of 0.2 MW.

The RFQ 102 may also be configured to operate as a beam output controller 120—when operated as a “chopper”, if there is no beam controller associated with the source, in which case a pulsed proton beam 115 may still be provided using a continuous proton source 110. The beam output controller function described above may then be partially or fully integrated into the RFQ 102, or control may be distributed between the RFQ 102 and the proton source 110.

The proton beam 115 exiting the first accelerating stage 102 enters the second accelerating stage 104. In this particular embodiment, the second stage 104 may be provided by one or more SCDTLs (Side Coupled Drift-Tube Linac) which accelerate the beam up to approximately 25 to 50 MeV, preferably 37.5 MeV. As an example, a suitable SCDTL 104 may operate at 3 GHz and four of these SCTDLs may be operated in cascade to achieve the 37.5 MeV acceleration.

The proton beam 115 exiting the second accelerating stage 104 enters the third accelerating stage 106, which comprises one or more cascaded accelerator units 130, 230, 330, 430.

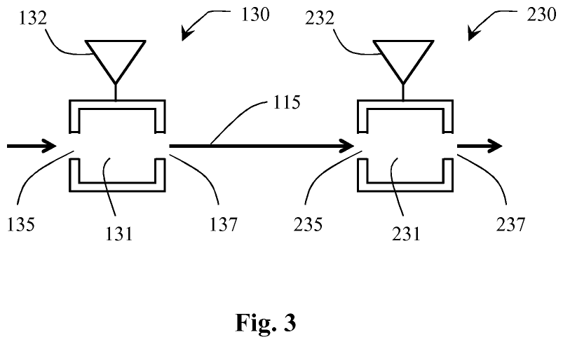

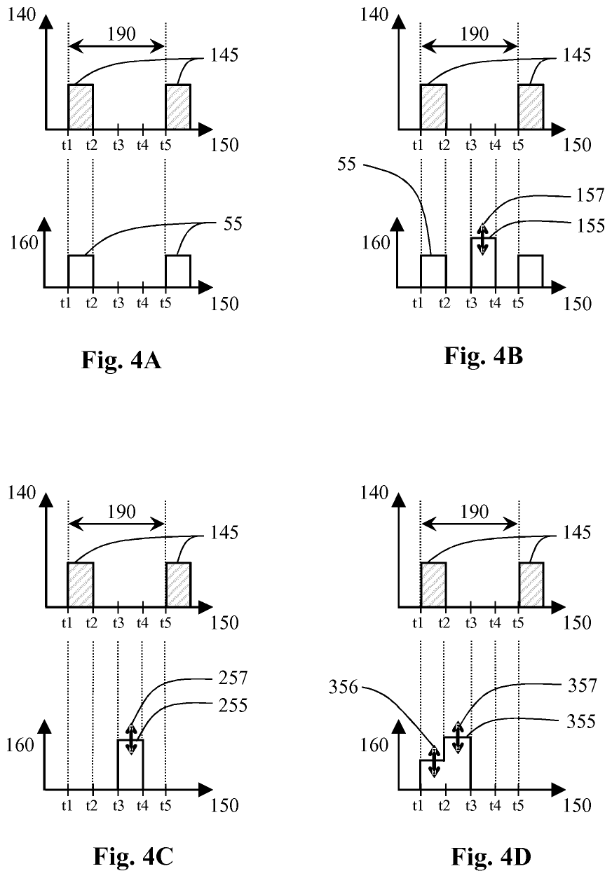

FIG. 2 depicts more details of the third accelerating stage 106 of FIG. 1 and FIG. 3 depicts two cascaded accelerating units 130, 230 in the third accelerating stage 106.

In this particular embodiment, the third stage 106 may be provided by one or more CCLs (Coupled Cavity Linac) 130, 230, 330, 430 which accelerate the beam up to the maximum energy of the system 100. This is approximately 50-300 MeV, and more typically in the range 70-250 MeV. As an example, a suitable CCL 130, 230, 330, 430 may operate at 3 GHz, and ten of these CCLs units may be operated in cascade to achieve the 230 MeV acceleration, each CCL providing 20 MeV acceleration.

Each accelerating unit 130, 230, 330, 430 comprising:
a proton beam input 135, 235 for receiving the proton beam 115;
a proton beam output 137, 237 for exiting the proton beam 115;
an RF energy source 132, 232, 332, 432 for providing RF energy during operation, such as a klystron;
at least one cavity 131, 231 extending from the proton beam input 135, 235 to the proton beam output 137, 237 for receiving RF energy from the RF energy source 132, 232 and for coupling the RF energy to the proton beam 115 as it passes from the proton beam input 135, 235 to the proton beam output 137, 237.

If more than one accelerating unit 130, 230 are cascaded as depicted in FIG. 3, the units are configured and arranged such that proton beam 115 exiting the proton beam output 137 of the upstream accelerating unit 130 may be received by the proton beam input 237 of the downstream accelerating unit 230.

The accelerator system 100 further comprises an RF energy controller 180 connected to one or more of the RF energy sources 132. The controller is configured and arranged to adjust the RF energy provided to the at least one cavity 131, 231. The controller 180 is further connected to the beam output controller 120, and further configured and arranged to provide RF energy from RF energy source 132, 232, 332, 432 during the off-time of the proton beam operating cycle 190.

The proton beam 115 may be delivered to the patient in therapeutic on-time pulses of a predetermined and/or controlled duration (typically between a few microseconds and a few milliseconds) at a predetermined and/or controlled repetition frequency (typically between 100 and 400 Hz). In cases where the therapeutic on-time is greater than the repetition period of the proton source 110, the proton beam duty cycle 145, 245 is the product of the therapeutic pulse on-time duration 145, 245 and the repetition frequency of the proton source 110. In cases where the therapeutic on-time is less than or equal to the repetition period of the proton source 110, the proton beam duty cycle 145, 245 is determined by the therapeutic pulse on-time duration 145, 245. The RF energy controller is configured and arranged to control one or more of the RF energy sources. They may be controlled independently or as a group. The RF energy sources 132, 232, 332, 432 may be operated at zero or maximum energy or at an intermediate energy value. Different energies in the proton beam 115 exiting the third accelerating stage 106 may thus be achieved by switching off the RF energy source 132, 232, 332, 432 of one or more accelerating units 130, 230, 330, 430.

If the accelerating units 130, 230, 330, 430 are configured substantially identically, the number of beam energy settings will be related to the number of accelerating units in the cascade. The beam energy in the proton beam 115 exiting the third accelerating stage 106 will correspond to the energy achievable by the last active accelerating unit 130, 230, 330, 430 in the cascade.

However, other configurations may also be used to provide intermediate acceleration values.

For example, accelerating units 130, 230, 330, 430 beyond the last active accelerating unit 130, 230, 330, 430 may be switched off, and further, the RF energy provided to the last active unit may be varied. The proton beam 115 exiting the third accelerating stage 106 may then have an intermediate energy which lies between the maximum energy producible by the last active accelerating unit and the energy producible by the previous accelerating unit.

This may be performed by modifying one or more of the characteristics of the RF energy emitted by the RF energy source 132, 232, 332, 432, such as RF amplitude, RF energy on-time, RF energy off-time, and/or RF energy pulse shape. Additionally or alternatively, degrading absorbers may also be used, or means to modify the geometry of the cavity and/or the RF coupling. For example, ferrite tuners or mechanical tuners may allow the module to be kept on resonance in spite of the temperature changes.

Additionally or alternatively, fine tuning of the energy may also be performed by modifying the phase of the final active accelerator unit 130, 230, 330, 430.

A combination of amplitude and phase variation (even several degrees) may limit degradation of the quality of the proton beam. By modifying the phase and/or the amplitude of the accelerating field, the proton beam 115 energy spread may be reduced.

The proton beam 115 which emerges from the third accelerating stage 106 is typically guided into a high energy beam transfer line, comprising bending magnets, to steer the beam into a nozzle for application to the patient during treatment.

The RF energy controller 180 is further configured to provide RF energy 132, 232, 332, 432 during the off-time of the proton beam operating cycle 190 for increasing or maintaining the temperature of the cavity 131.

The invention is based on the insight that the instability seen after accelerating units are turned on or off is mainly related to the temperature changes in the cavity 131, 231, 331, 431. Such cavities are typically made of metal, and substantial changes in RF power supplied to the cavity produce temperature changes which cause either contraction or expansion of the cavity. As the cavity supports tuned electromagnetic waves, any thermal expansion or contraction will tune the cavity off-resonance and disrupt the proton beam 115.

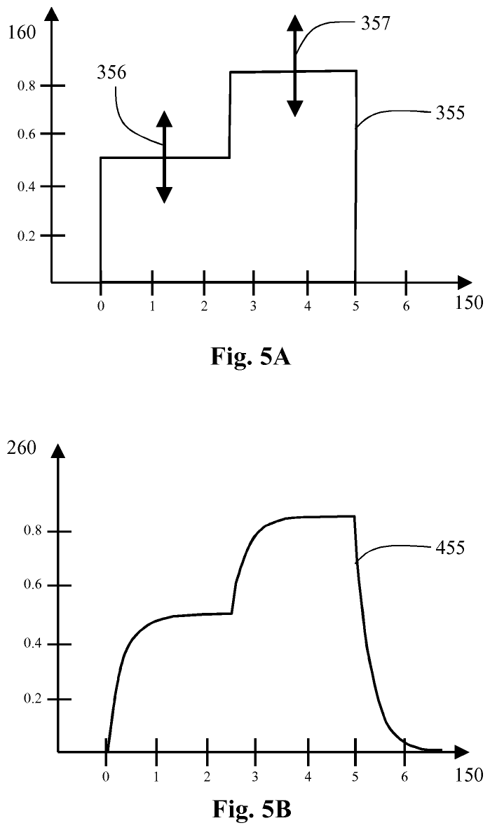

FIG. 4A depicts an example of operation of an accelerating unit 130, 230, 330, 430 in a conventional accelerating mode.

The upper graph plots a simplified view of the proton beam current 140 over a period of time 150 which includes five instants—t1, t2, t3, t4 and t5. The proton beam operating cycle 190 is depicted as running from t1 to t5, which is also the time between the start of two successive on-time pulses 145. Although the intervals between the instants are depicted approximately equal, this may not be the case in practice—they may even vary by orders of magnitude. The pulses are depicted schematically as square wave pulses, but the actual waveforms may have a non-negligible rise and fall-time.

The beam current rises from zero to its maximum at instant t1 and back to zero at t2 for the on-time of this proton beam operating cycle 190, the pulse 145 being of approximately uniform amplitude. During the rest of the proton beam operating cycle 190, including intervals t2 to t3, t3 to t4 and t4 to t5, the beam current (and the beam energy) is zero, or approximately zero. In other words, the off-time for the proton beam is from t2 to t5. Starting at t5, the proton beam operating cycle 190 repeats with the successive on-time proton beam pulse 145.

The lower graph of FIG. 4A plots a simplified view of the RF energy 160 provided by the RF energy source 132, 232 over the same period of time 150 with the same instants. The RF energy rises from zero to an acceleration peak value at t1 and back to zero at t2, this first RF energy pulse 55 being of approximately uniform amplitude. During the rest of the proton beam operating cycle 190, including intervals t2 to t3, t3 to t4 and t4 to t5, the RF energy is zero, or approximately zero. Starting at t5, the proton beam operating cycle 190 repeats and the successive first RF energy pulse 55 is provided due to the synchronization of the RF energy pulses 55 with the proton beam operating cycle 190.

The duration of the first RF energy pulse 55 from t1 to t2 and the acceleration field peak value are predetermined and/or controlled to provide the desired acceleration of the proton beam by the RF energy during the proton beam on-time pulse. Acceleration occurs between t1 and t2.

In practice, the first RF energy pulse 55 may be varied for different proton beam operation cycles 190 to provide variable acceleration and consequently variable proton beam energy. The inventors have determined that operating the accelerating units at different RF energy levels may change the temperature, and thus the resonant frequency of the cavities 131, 231. This off resonance operation of a cavity 131, 231 may mean that the proton beam energy is not as planned, resulting in a disruption in the optimum treatment plan.

The accelerating units according to the invention may be used in two types of operating mode: non-accelerating, where the accelerating unit passes the proton beam 115 through with no substantial acceleration, and an accelerating mode, where the proton beam is substantially accelerated.

FIG. 4B depicts an example of operation of an accelerating unit 130, 230, 330, 430 in an improved accelerating mode. The upper graph is identical to the upper graph of FIG. 4A depicting a similar proton beam operating cycle 190.

The lower graph of FIG. 4B plots the RF energy 160 over the same period of time 150 with the same instants t1, t2, t3, t4 and t5. The first RF energy pulse 55 is provided between t1 and t2 as depicted in FIG. 4A and is of approximately uniform amplitude. The RF energy remains at zero, or approximately zero, in the interval t2 to t3. The RF energy then rises from zero to a first compensation peak value 157 at t3 and back to zero at t4, forming a first RF energy compensation pulse 155 being of approximately uniform amplitude 157. During the rest of the proton beam operating cycle 190, the RF energy is zero, or approximately zero. Starting at t5, the proton beam operating cycle 190 repeats and the successive first RF energy pulse 55 is provided as depicted in FIG. 4A.

The interval between the end of the first RF acceleration pulse 55 and the start of the first RF compensation pulse 155, depicted here as t2 to t3, may be any convenient value. The first compensation peak value 157 may be selected to be substantially equal to the peak value of the first RF acceleration pulse 55, or it may be lower, or it may be higher.

The duration of the first RF energy pulse 55, from t1 to t2, and the acceleration peak value are predetermined and/or controlled to provide the desired acceleration of the proton beam by the RF energy during the proton beam on-time. Acceleration occurs between t1 and t2.

The duration of the RF energy compensation pulse, from t3 to t4, and the compensation peak value 157 are predetermined and/or controlled to compensate for the temperature change which may be expected when the accelerating unit is operated in acceleration mode at a reduced RF energy acceleration level compared to an earlier RF energy acceleration level. The compensation RF energy pulse 155 does not substantially overlap in time with the proton beam current pulse 145. In FIG. 4B, the proton beam pulse 145 and the compensation pulse 155 are separated, in time, by interval t2 to t3 of zero, or approximately zero, RF energy. This interval t2 to t3 may be selected to minimize, or even eliminate, acceleration due to the application of a portion of the first RF energy compensation pulse 155 during any portion of the on-time 145 of the proton beam 145. In practice, the on-time of the proton beam 145, here from t1 to t2, is typically measured in microseconds, and the interval between beam pulses is typically measured in milliseconds.

PCT application WO 2018/043709 A1 teaches that, at least for semiconductor applications, heating of the cavities due to higher order modes may be reduced by randomizing the proton beam current pulse period using a randomized laser on/off pattern. This application teaches away from heating of cavities for any purpose. No mention is made of modulating the RF energy for any purpose.

PCT application WO 2015/175751 A1 exclusively describes electron acceleration, so it provides no teaching suitable for proton acceleration. It discloses embodiments configured to generate X-rays with dual energies for cargo inspection, so they cannot provide a teaching that is relevant for irradiating tissue with protons. Additionally, no mention is made of the heating of cavities.

FIG. 4C depicts an example of operation of an accelerating unit 130, 230, 330, 430 in an improved non-accelerating mode. The upper graph is identical to the upper graph of FIGS. 4A and 4B depicting a similar proton beam operating cycle 190.

The lower graph of FIG. 4C plots the RF energy 160 over the same period of time 150 with the same instants t1, t2, t3, t4 and t5.

However, in this embodiment, no RF acceleration energy pulse is provided—during interval t1 to t2 (during the proton beam 145 on-time) the RF energy is zero, or approximately zero. The RF energy rises from zero to a second compensation peak value 257 at t3 and back to zero at t4, this RF energy compensation pulse 255 being of approximately uniform amplitude. During the rest of the proton beam operation cycle 190, the RF energy is zero, or approximately zero.

The duration of the RF energy compensation pulse 255, from t3 to t4, and the compensation peak value 257 are predetermined and/or controlled to compensate for the temperature change which may be expected when the accelerating unit is operated in non-accelerating mode for one or more proton beam operation cycles 190 after a period of acceleration. In the non-accelerating mode, the compensation RF energy pulse 255 does not substantially overlap in time with the proton beam current pulse 145. In FIG. 4C, the proton beam pulse 145 and the compensation pulse 255 are separated, in time, by interval t2 to t3 of zero, or approximately zero, RF energy. This interval t2 to t3 may selected to minimize, or even eliminate, acceleration due to the application of a portion of the RF energy compensation pulse 255 during any portion of the on-time 145 of the proton beam 115.

Preferably, the expected temperature change is fully compensated, but if this is not possible due to operating constraints, partially compensating for the temperature change is still advantageous compared to the situation known in the prior art.

The skilled person will realize that the waveforms depicted in FIG. 4 are schematic, and the actual waveforms may have a non-negligible rise and fall-time which may need to be taken into account when determining the control parameters used. Similarly, slight beam current variations may also need to be taken into account.

The skilled person will also realize that any RF energy waveform shape is possible, not just the square-wave pulses 55, 155, 255 depicted. For example, a triangular or ramp-shape.

Providing an RF compensation pulse 155, 255, 355 during the off-time of the proton beam may also be advantageous when successive RF energy acceleration pulses 55, 356 provide similar or identical power. Following off-time, a cavity 131, 231 may need a short period of time to settle once an RF energy acceleration pulse 55, 356 has been applied. This instability may limit the usable proton beam pulse 145 as an excessive instability in the energy of the proton beam pulses 145 may result in positioning instability of the proton beam during operation. By providing appropriate RF compensation pulses 155, 255, 355 during the proton beam off-time, this settling time may be reduced, or even eliminated.

The energy controller 180 may be configured to provide substantially the same or substantially different RF pulses to each accelerator unit during a particular proton beam operation cycle 190. The accelerator units may be operated individually or in groups. The RF pulses to an individual accelerator unit may also vary during the operation of the system 100 over more than one proton beam operation cycle 190. This provides a very flexible and accurate system to control and stabilize beam energy variation caused by the accelerator system 100 itself, or external disruptive elements.

FIG. 4D depicts a further example of operation of an accelerating unit 130, 230, 330, 430 in improved accelerating mode. The upper graph is identical to the upper graph of FIGS. 4A, 4B and 4C depicting a similar proton beam operating cycle 190.

The lower graph of FIG. 4D plots the RF energy 160 over the same period of time 150 with the same instants t1, t2, t3, t4 and t5. A complex RF energy pulse 355 is provided—the RF energy rises from zero to a complex acceleration peak value 356 at t1, the RF energy pulse 355 being of approximately uniform amplitude between t1 and t2. At t2, the RF energy rises from the complex acceleration peak value 356 to a complex compensation peak value 357 at t2 and back to zero at t3, the RF energy pulse 255 being of approximately uniform amplitude between t2 and t3. During the rest of proton beam operation cycle 190, the RF energy is zero, or approximately zero. The RF energy is approximately a step-shaped pulse 355.

The duration of the complex RF energy pulse 355 from t1 to t2 and the complex acceleration peak value 356 are predetermined and/or controlled to provide the desired acceleration of the proton beam by the RF energy during the proton beam on-time 145. Acceleration occurs between t1 and t2.

The duration of the complex RF energy pulse 355 from t2 to t3, and the complex compensation peak value 357 are predetermined and/or controlled to compensate for the temperature change which may be expected when the accelerating unit is operated in acceleration mode after one or more intervals of non-acceleration.

The compensation portion of the RF energy pulse 355 as depicted appears to overlap in time with the proton beam current pulse 145. However, the skilled person will realize that the rise time of the complex compensation peak value 357 may be delayed slightly to reduce disruption to the energy of the proton beam 115.

In practice, the compensation peak value 257,357 may be higher, equal or lower than the acceleration peak value 256, 356. Preferably, the expected temperature change is fully compensated, but if this is not possible due to operating constraints, partially compensating for the temperature change is still advantageous compared to the situation known in the prior art.

The skilled person will also realize that any RF energy waveform shape is possible, not just the step-wave pulse 355 depicted. The acceleration level 256, 356 may higher, equal or lower than the compensation level 257, 357.

As mentioned previously, the accelerating unit may be operated in a maximum energy on or off modes, or an intermediate RF energy level may be assigned.

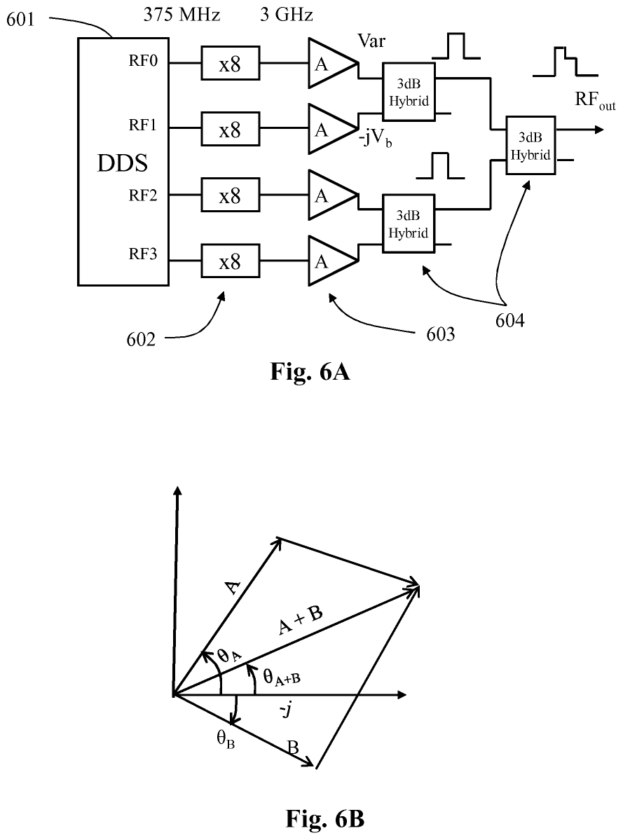

FIG. 5 depicts further details of the improved operation depicted in FIG. 4D. FIG. 5A shows the RF energy 160 supplied to a cavity over 0 to 6 microseconds. The complex RF energy 355 is provided—the RF energy pulse 355 rises from zero to the complex acceleration peak value 356 of 0.5 units at 0 microseconds. The RF energy then rises to the complex compensation peak value 357 of 0.8 units at approximately 2.5 microseconds and back to zero at 5 microseconds. During the rest of the proton beam operation cycle 190, the RF energy is zero, or approximately zero. The RF energy is approximately a step-shaped pulse 355. The units depicted here (0 to 0.8) on the vertical axis are nominal units.

The duration of the RF energy pulse 355 from 0 to 2.5 and the complex acceleration peak value 356 are predetermined and/or controlled to provide the desired acceleration of the proton beam by the RF energy during the proton beam on-time. Acceleration occurs between 0 and 2.5 microseconds. The duration of the RF energy pulse, 2.5 to 5 microseconds, and the complex compensation peak value 357 are predetermined and/or controlled to compensate for the temperature change which will be expected when the accelerating unit is operated in acceleration mode after one or more intervals of non-acceleration.

FIG. 5B depicts the accelerator field intensity 260 in an accelerator unit cavity 131, 231 over the same period of time 150. The accelerator field 455 rises from zero at 0 microseconds to a first level (of approximately 0.5 units) determined by the RF acceleration peak value 256 with a slight lag. The first level is reached at about 1 microsecond. At about 2.5 microseconds, the accelerator field starts to rise to a second level (of approximately 0.8 units) determined by compensation peak value 257 with a slight lag. It reaches the second level at about 3.5 microseconds. At 5 microseconds, the value drops towards zero, reaching 0 at approximately 6.5 microseconds. The accelerator field rises from zero at 0 microseconds to a first level and then further to a second level, creating a distorted step-shaped pulse 455 compared to the RF energy pulse 355. The units depicted here (0 to 0.8) on the vertical axis are nominal units.

The differences between FIGS. 5A and 5B represent the accelerator cavity response to the RF energy waveform, and this should preferably be taken into account when determining, for example, the most suitable input RF energy values and durations to compensate for the temperature change and the settling time to be compensated for. For example, a lag in response of the accelerator field to a rise in input RF energy to the complex compensation value 357 may limit, or even avoid, disruption to the energy of final portion of the proton beam pulse 145 which occurs at the same time as the complex acceleration portion of the complex RF energy 355. Such characteristics may be found in product documentation or measured in a test environment or during operation with appropriate sensors.

The peak RF power produced by the RF energy source, such as a klystron, consists of two components, the power dissipated in the cavity and the power transferred to the beam. Although in medical applications the peak beam current is low, typically 300 uA, it may be advantageous to account for this by overcoupling the cavity.

If the power dissipated in the cavity at full energy is P_cav_max and the power dissipated at reduced power is P_cav1, the energy U0 deposited in the cavity at full energy is:

U0=P_cav_max x the pulse width t,

with the appropriate corrections for the power lost during the cavity fill and decay times. The energy deposit during the reduced amplitude pulse is U1.

To prevent significant changes in cavity temperature, an additional amount of energy must be supplied within a time short compared to the thermal response time of the cavity. This may be done on a pulse-by-pulse basis, or the additional energy may be supplied on a longer time scale, subject to the constraint that the cavity frequency fluctuations are small enough not to affect the performance of the accelerator significantly.

If the cavity energy supplied during an active beam pulse is:

U1=P_cav1*t,

the additional energy that must be supplied is:

U2=(P_cav_max−P_cav1)*t.

This energy U2 may be provided with any peak power and pulse length subject to the constraint that the total energy is U2, such that, averaged over times short compared to the thermal response time of the cavity, the total power dissipation, and thus the cavity temperature is substantially constant—in other words, constant within an acceptable tolerance, preferably a few tens of degree.

It may also be advantageous to provide substantially the same RF energy 132 for each successive proton beam operating cycle 190. This provides a substantially constant average RF power to the cavity during operation, increasing the proton beam energy stability over more than one operating cycle 190.

FIG. 7A depicts the synchronization of three RF energy control configurations 701, 702, 703 that keep the average power substantially constant by providing separate RF energy pulses during both the proton beam on-time and off-time. The proton beam operating cycle 190 is also depicted to illustrate the synchronization of the RF energy control with the proton beam operating cycle 190.

Four waveforms are depicted over two operating cycles 190 of the proton beam pulse 245, including nine instants—t1, t2, t3, t4, t5, t6, t7, t8, t9. These instants are depicted symmetrically, but in practice the intervals between the instants may vary considerably. They are used here in the same way as for FIG. 4—to schematically explain the synchronization.

For a typical operation of 100 pulses per second, or 100 Hz, the period of the operating cycle 190 is 10 milliseconds. An operation cycle 190 of 25% on-time and 75% off-time is depicted, which is also called a 25% or 1:3 duty cycle. In practice, however, any suitable ratio may be used.

The top waveform 700 depicts the proton beam pulses 245 during the two operating cycles 190. The beam current rises from zero to its maximum at instant t1 and back to zero at t2 for the on-time of this first beam operating cycle 190, the pulse 245 being of approximately uniform amplitude. Between t2 to t5, the beam current (and beam energy) is zero, or approximately zero, for the off-time of this first beam operating cycle 190. The waveform repeats during the second operating cycle 190, with maximum beam current between t5 & t6 and zero, or approximately zero, beam current (and beam energy) between t6 & t9.

The first RF control configuration graph 701 plots the RF energy provided to an acceleration unit 130, 230 330, 430 over the same period of time. At the start of the first operating cycle 190, the RF energy rises from zero to a reference acceleration peak value at t1 and back to zero at t2, the RF energy pulse being of approximately uniform amplitude. During the rest of this first operating cycle 190, including instants t3 and t4, the RF energy is zero, or approximately zero. The waveform repeats during the second operating cycle 190, with the reference acceleration peak value between t5 & t6, and zero, or approximately zero, RF energy between t6 & t9.

The duration of the RF energy pulse from t1 to t2 and t5 to t6 and the reference acceleration peak value are predetermined and/or controlled to provide the desired acceleration of the proton beam by the RF energy during the proton beam on-time. Acceleration occurs between t1 & t2 and t5 & t6. This RF control configuration is the reference for the other two configurations 702, 703, so the reference acceleration peak value is considered here to be nominally 100%. During operation according to 701, the RF energy is provided to the cavity in a single pulse per proton beam operating cycle 190 at substantially the same time as the on-time of the proton beam.

The second RF control configuration graph 702 plots the RF energy over the same period of time. At the start of the first operating cycle 190, the RF energy rises from zero to a first acceleration peak value at t1 and back to zero at t2, the RF energy pulse being of approximately uniform amplitude. This first acceleration peak value is approximately 75% of the reference acceleration peak value depicted in graph 701. The RF energy rises from zero to a first compensation peak value at t3 and back to zero at t4. This first compensation peak value is approximately 25% of the reference acceleration peak value depicted in graph 701. During the rest of this first operating cycle 190, the RF energy is zero, or approximately zero. The waveform repeats during the second operating cycle 190, with an acceleration peak value between t5 & t6 and a compensation peak value between t7 & t8.

The duration of the RF energy pulses from t1 to t2 and t5 to t6 and the first acceleration peak value are predetermined and/or controlled to provide the desired acceleration of the proton beam by the RF energy during the proton beam on-time. Acceleration occurs between t1 & t2 and t5 & t6.

In general, the duration of the RF energy pulses, t3 to t4 and t7 to t8, and the first compensation peak value are predetermined and/or controlled to compensate for the temperature change which would be expected when the accelerating unit is operated with a lower acceleration peak value compared to previous operating cycles. During operation, the RF energy is provided to the cavity in two pulses per proton beam operating cycle 190—the first at substantially the same time as the on-time of the proton beam, and the second at substantially the same time as the off-time of the proton beam.

In this particular example, 702, the pulse durations of the compensation and acceleration pulses are the same, so by ensuring that the peak values of the uniform amplitude compensation and acceleration pulses add up to 100% of the reference peak value 701, the RF energy provided to the cavity for each successive operating cycle 190 is substantially the same in both 702 and 701.

The third RF control configuration graph 703 plots the RF energy over the same period of time and is very similar to the second RF control configuration 702. The third configuration 703 also provides an acceleration pulse of uniform amplitude between t1 & t2 during the beam on-time and a compensation pulse of uniform amplitude between t3 & t4 during the first operating cycle. This is repeated in the second operating cycle 190 with an acceleration pulse of uniform amplitude between t5 & t6 and a compensation pulse of uniform amplitude between t7 & t8.

The third configuration 703 differs from the second 702 in the peak values. Here the acceleration pulses have a second acceleration peak value of approximately 50% of the reference acceleration peak value depicted in graph 701. Similarly, the compensation pulses have a second compensation peak value of approximately 50% of the reference acceleration peak value depicted in graph 701.

The duration of the RF energy pulses from t1 to t2 and t5 to t6 and the second acceleration peak value are predetermined and/or controlled to provide the desired acceleration of the proton beam by the RF energy during the proton beam on-time. Acceleration occurs between t1 & t2 and t5 & t6. In general, the duration of the RF energy pulses, t3 to t4 and t7 to t8, and the second compensation peak value are predetermined and/or controlled to compensate for the temperature change which would be expected when the accelerating unit is operated with a lower acceleration peak value compared to previous operating cycles. During operation, the RF energy is provided to the cavity in two pulses per proton beam operating cycle 190—the first at substantially the same time as the on-time of the proton beam, and the second at substantially the same time as the off-time of the proton beam.

In this particular example, 703, the pulse durations of the compensation and acceleration pulses are the same, so by ensuring that the peak values of the uniform amplitude compensation and acceleration pulses add up to 100% of the reference peak value 701, the RF energy provided to the cavity for each successive operating cycle 190 is substantially the same in both 703 and 701. It is also substantially the same as in the second configuration 702.

So substantially constant average power may be achieved by interspersing the compensating pulses, during the proton beam off-time, between the accelerating pulses, during the proton beam on-time 245. The time between RF energy pulses are preferably short compared to the thermal time response of the cavity. The amplitude of the first pulse may be varied over the full range from maximum power to nearly zero power. Likewise, the power in the second pulse may be varied from maximum power to nearly zero power to keep the average power substantially constant. A further advantage of this approach may be that the total average power required is substantially less than in prior art systems. In some cases, it may even be nearly half that required in systems without this substantially constant average power feature.

For a typical klystron modulator and power supply, the nominal RF pulse width available for accelerating the beam may be 5 microsecond flattop, and power supplies may limit operation to 200 pulse per second, or 200 Hz.

To implement the substantially constant average power configuration, within the constraints imposed by such typical modulator specifications, it may be advantageous to divide each 5 μs pulse into two intervals of approximately 2 to 2.5 microseconds each (as depicted in FIG. 5A). The stepped pulse is predetermined and/or controlled to have the same area under the power curve as the 5 microsecond flattop.

During the first pulse interval, the RF power is set to the complex acceleration peak value. The proton beam current is turned on during that interval, and the beam current is increased so that the total charge accelerated is the same as with the full 5 microsecond interval without the substantially constant power feature. Because the beam current is so low, this is expected to have a negligible effect on the peak power required.

During the second RF pulse interval, the proton beam is turned off and the RF power level, and possibly the pulse length, may be adjusted to provide the energy required to keep the average RF power substantially constant.

This means that the power dissipation in the accelerator may remain substantially constant, and thus the temperature of the full accelerator will also stay substantially constant while changing the energy of the beam by using one accelerator unit or a sequence of accelerating units.

The amplitude of the first pulse interval may be varied over the full range from maximum power to nearly zero power. Likewise, the power in the second pulse interval may be varied from maximum power to nearly zero power to keep the average power substantially constant.

FIG. 7B depicts two further RF energy control configurations 704, 705 that keep the average power substantially constant using two pulse intervals. However, these do it by dividing each RF pulse into two intervals, one interval being provided during the proton beam on-time 245 and the other interval during the proton beam off-time.

The duration depicted is the same as for FIG. 7A, and the reference acceleration peak value of 100% is also the same. For convenience, the same two operating cycles 190 of the proton beam pulses 245 of FIG. 7A are also depicted as the top waveform 700. In addition, the first RF control configuration 701 of FIG. 7A is repeated as the first RF control configuration using the reference acceleration peak value of 100%.

For operation at higher proton pulse rates, it may be more convenient to provide a single pulse with two intervals. For a typical operation of 200 pulses per second, or 200 Hz, the period of the operating cycle 290 is 5 milliseconds. An operating cycle 190 of 25% on-time and 75% off-time is depicted, which is also called a 25% or 1:3 duty cycle. In practice, however, any suitable ratio may be used.

The fourth RF control configuration graph 704 plots the RF energy over the same period of time. At the start of the first operating cycle 190, the RF energy rises from zero to a third acceleration peak value at t1, changes to a third compensation peak value at t2 and drops back to zero at t3, the RF energy pulse comprising two intervals of approximately uniform amplitude. This third acceleration peak value is approximately 75% of the reference acceleration peak value depicted in graph 701. This third compensation peak value is approximately 25% of the reference acceleration peak value depicted in graph 701. During the rest of this first operating cycle 190, the RF energy is zero, or approximately zero. The waveform repeats during the second operating cycle 190, with an acceleration peak value between t5 & t6 and a compensation peak value between t6 & t7.

The duration of the RF energy pulse interval from t1 to t2 and t5 to t6 and the third acceleration peak value are predetermined and/or controlled to provide the desired acceleration of the proton beam by the RF energy during the proton beam on-time. Acceleration occurs between t1 & t2 and t5 & t6.

In general, the duration of the RF energy pulse interval from t2 to t3 and t6 to t7, and the third compensation peak value are predetermined and/or controlled to compensate for the temperature change which would be expected when the accelerating unit is operated with a lower acceleration peak value compared to previous operating cycles. During operation, the RF energy is provided to the cavity in a single pulse per proton beam operating cycle 190, the pulse being divided into two intervals—the first interval at substantially the same time as the on-time of the proton beam 245, and the second interval at substantially the same time as the off-time of the proton beam.

In this particular example, 704, the durations of the compensation and acceleration pulse intervals are the same, so by ensuring that the peak values of the uniform amplitude compensation and acceleration pulses add up to 100% of the reference peak value 701, the RF energy provided to the cavity for each successive operating cycle 190 is substantially the same in both 704 and 701. Similarly, it is also substantially the same as in 702 and 703.

The fifth RF control configuration graph 705 plots the RF energy over the same period of time and is very similar to the fourth RF control configuration 704. The fifth configuration 705 also provides a pulse with two intervals—an acceleration pulse interval of uniform amplitude between t1 & t2 during the proton beam on-time 245 and a compensation pulse interval of uniform amplitude between t2 & t3 during the first operating cycle 190. This is repeated in the second operating cycle 190 with an acceleration pulse interval of uniform amplitude between t5 & t6 and a compensation pulse interval of uniform amplitude between t6 & t7.

The fifth configuration 705 differs from the fourth 704 in the peak values of the intervals. Here the acceleration pulse intervals have a fourth acceleration peak value of approximately 50% of the reference acceleration peak value depicted in graph 701. Similarly, the compensation pulse intervals have a fourth compensation peak value of approximately 50% of the reference acceleration peak value depicted in graph 701.

The duration of the RF energy pulse intervals from t1 to t2 and t5 to t6 and the fourth acceleration peak value are predetermined and/or controlled to provide the desired acceleration of the proton beam by the RF energy during the proton beam on-time 245. Acceleration occurs between t1 & t2 and t5 & t6. In general, the duration of the RF energy pulse intervals t2 to t3 and t6 to t7, and the fourth compensation peak value are predetermined and/or controlled to compensate for the temperature change which would be expected when the accelerating unit is operated with a lower acceleration peak value compared to previous operating cycles. During operation, the RF energy is provided to the cavity in two pulse intervals per proton beam operating cycle 190—the first interval at substantially the same time as the on-time of the proton beam, and the second interval at substantially the same time as the off-time of the proton beam.

In this particular example, 705, the pulse durations of the compensation and acceleration pulse intervals are the same, so by ensuring that the peak values of the uniform amplitude compensation and acceleration pulse intervals add up to 100% of the reference peak value 701, the RF energy provided to the cavity for each successive operating cycle 190 is substantially the same in both 704 and 701. It is also substantially the same as in the other configurations 702 and 703.

So substantially constant average power may also be achieved by interspersing the compensating pulse intervals during the proton beam off-time, between the accelerating pulse intervals during the proton beam on-time 245. The time between RF energy pulses are preferably short compared to the thermal time response of the cavity.

The compensating pulses may even have a lower peak value and a longer pulse duration than the examples above. However, this approach requires a more powerful modulator since the average klystron cathode current will increase.

For some embodiments, the RF power level may need to be switched quickly in a short time compared to the cavity response time by having a dual source and simply switching from one to the other. It may even need to be performed within a few ns.

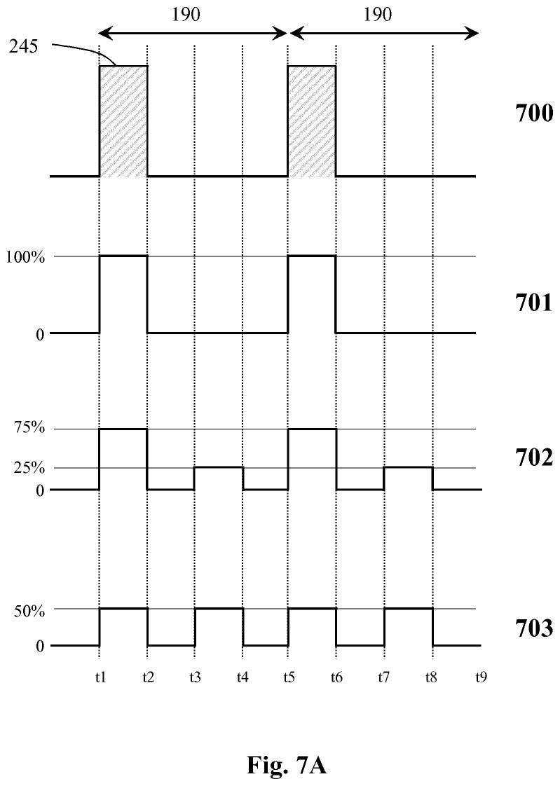

A block diagram of a suitable low-level RF unit employing a DDS chip is shown in FIG. 6A. In the preferred embodiment, the dual source is an Analog Devices AD9959 Direct Digital Synthesis (DDS) chip 601 which has four output channels RF0, RF1, RF2, RF3. As the required 3 GHz frequency cannot usually be generated directly, 375 MHz may be generated in all four channels RF0, RF1, RF2, RF3. Each channel comprises an 8× frequency multiplier chain with a cascade of three full wave frequency doublers 602, bandpass filters and amplifiers 603. The outputs of two channels are combined using suitable RF couplers 604, such as Hybrid 3 dB. The phase of each channel is set to give the desired output phase and amplitude for the desired energy. All channels have a gate input that turns the output signal on and off with a fast rise and fall time and a short (few ns) delay. Channel 0 and 1 are turned on simultaneously to yield the output for the first-time interval 1, while channels 2 and 3 remain off.

At the end of time interval 1 the beam and channels 0 and 1 of the DDS unit are turned off, and channels 2 and 3 are turned on. Channels 2 and 3 have previously had their phases set to provide the desired amplitude and phase for the second interval. Amplitude adjustment of the RF output signal RFout does not affect phase.

In practice, it may be advantageous to keep the phase during the second interval the same as the phase during the first interval. Since there is no proton beam to disrupt, the phase during the second interval may be ignored. However, if the phases are configured to match, it may allow a quicker change in amplitudes from one pulse, or pulse interval, to the next. Having different phases may cause a spike or dip in the cavity field amplitude that may increase the time required to reach the new level for the second interval. Additionally, it may also have an effect on the overall temperature of the accelerating unit.

FIG. 6B depicts the phasor diagram of the two signals which may be used to modulate the amplitude and phase of the RF drive envelope made of two adjacent pulses. Amplitude varies with θA-θB. Phase varies with θA+θB.

In practice each accelerator unit may also have a separate, local DDS unit. The DDS units are operated at substantially the same frequency and are phase synchronized with all the other units in the accelerator system.

The invention is not limited to the use of the DDS technology: many possibilities for frequency generation are open to a designer, ranging from phase-locked-loop to dynamic programming of digital-to-analog converter outputs to generate arbitrary waveforms.

Here the choice has been made for a DDS technique because of its high resolution and accuracy being a single-chip IC device which may generate programmable analog output waveforms.

The accelerator units may be any suitable RF linear accelerator (or Linac), such as a Coupled Cavity Linac (CCL), a Drift Tube Linac (DTL), a Separated Drift-Tube Linac (SDTL), a Side-Coupled Linac (SCL), or a Side-Coupled Drift Tube Linac (SCDTL). They may all be the same type, or different types may be combined in cascade.

It will be appreciated that the invention—especially many of the method steps indicated above—also extends to computer programs, particularly computer programs on or in a carrier, adapted for putting the invention into practice. The program may be in the form of source code, object code, a code intermediate source and object code such as partially compiled form, or in any other form suitable for use in the implementation of the method according to the invention.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. Use of the verb “comprise” and its conjugations does not exclude the presence of elements or steps other than those stated in a claim. The article “a” or “an” preceding an element does not exclude the presence of a plurality of such elements. The invention may be implemented by means of hardware comprising several distinct elements, and by means of a suitably programmed computer. In the system claim enumerating several means, several of these means may be embodied by one and the same item of hardware. The mere fact that certain measures are recited in mutually different dependent claims does not indicate that a combination of these measures cannot be used to advantage.

### REFERENCE NUMBERS

55 first RF energy accelerating pulse
100 proton linear accelerator system
102 first accelerating stage, e.g. Radio-Frequency Quadrupole (RFQ)
104 second accelerating stage, e.g. Side-Coupled Drift Tube Linac (SCDTL)
106 third accelerating stage, e.g. Coupled Cavity Linac (CCL)
110 proton source
115 proton beam
120 beam output controller
130 first accelerator unit
131 first cavity
132 first RF energy source
135 first proton beam input
137 first proton beam output
140 axis: beam current (FIG. 4)
145 proton beam operating cycle
150 axis: period of time (FIGS. 4 & 5)
155 first RF energy compensation pulse
157 first RF compensation pulse interval peak value
160 Axis: RF energy (FIGS. 4 & 5A)
180 RF energy controller
190 proton beam operating cycle [FIGS. 7A & 7B]
230 second accelerator unit
231 second cavity
232 second RF energy source
235 second proton beam input
237 second proton beam output
245 proton beam pulse or duty cycle
255 second RF energy compensation pulse
257 second RF compensation pulse interval peak value
260 axis: accelerator field intensity in cavity (FIG. 5B)
330 third accelerator unit
332 third RF energy source
355 complex RF energy pulse (acceleration interval & compensation interval)
356 complex RF acceleration pulse interval peak value
257 complex RF compensation pulse interval peak value
430 fourth accelerator unit
432 fourth RF energy source
455 Accelerator field (FIG. 5B)
601 DDS chip
602 cascade of three full wave frequency doublers
603 amplifiers
604 RF couplers
700 proton beam pulses during two operating cycles
701 first RF control configuration
702 second RF control configuration
703 third RF control configuration
704 fourth RF control configuration
705 fifth RF control configuration

1. A proton linear accelerator system for irradiating tissue, the accelerator system comprising:
a proton source for providing a proton beam during operation;
a beam output controller for adjusting the beam current of the proton beam exiting the source;
a first accelerator unit having:
a first proton beam input for receiving the proton beam;
a first proton beam output for exiting the proton beam;
a first RF energy source for providing RF energy during operation;
at least one first cavity extending from the first proton beam input to the first proton beam output, for receiving RF energy from the first energy source and for coupling the RF energy to the proton beam as it passes from the first beam input to the first beam output;
the system further comprising:
an RF energy controller connected to the first RF energy source for adjusting the RF energy provided to the at least one first cavity and further connected to the beam output controller;
the beam output controller being configured to provide proton beam pulses with a predetermined and/or controlled beam operating cycle; and
the RF energy controller being configured to provide RF energy during the off-time of the proton beam operating cycle such that the temperature of the first cavity is increased or maintained.

2. The accelerator system according to claim 1, wherein the RF energy controller is further configured to provide substantially the same RF energy for each successive proton beam operating cycle.

3. The accelerator system according to claim 1, wherein the RF energy controller is further configured to provide RF energy during both the on-time and the off-time of the proton beam operating cycle.

4. The accelerator system according to claim 1, wherein the system further comprises:
a second accelerator unit having:
a second proton beam input for receiving the proton beam from the first accelerator unit;
a second proton beam output for exiting the proton beam;
a second RF energy source for providing RF energy during operation;
at least one second cavity extending from the second proton beam input to the second proton beam output, for receiving RF energy from the second energy source and for coupling the RF energy to the proton beam as it passes from the second beam input to the beam output;
the RF energy controller being further connected to the second RF energy source for adjusting the RF energy provided to the at least one second cavity; and
the RF energy controller being configured to provide RF energy during the off-time of the proton beam operating cycle such that the temperature of the second cavity is increased or maintained.

5. The accelerator system according to claim 4, wherein the RF energy provided to the first and second cavities is substantially the same.

6. The accelerator system according to claim 1, wherein the RF energy controller is configured to provide a predetermined and/or controlled energy by modifying one or more of the following characteristics of the RF energy:
RF amplitude, RF energy on-time, RF energy off-time, RF energy pulse shape or any combination thereof.

7. The accelerator system according to claim 1, wherein the first accelerator unit and/or second accelerator unit are of one of the following types:
Coupled Cavity Linac (CCL), Drift Tube Linac (DTL), Separated Drift-Tube Linac (SDTL), Side-Coupled Linac (SCL), Side-Coupled Drift Tube Linac (SCDTL).

8. A method of operating a proton beam suitable for irradiating tissue, the method comprising:
providing proton beam pulses with a predetermined and/or controlled beam operating cycle from a proton beam source;
adjusting the beam current of the proton beam exiting the source;
providing RF energy from a first RF energy source to at least one first cavity;
coupling the RF energy to the proton beam as it passes through the at least one cavity; and
adjusting the RF energy provided to the at least one first cavity to provide RF energy during the off-time of the proton beam operating cycle such that the temperature of the first cavity is increased or maintained.

9. The method according to claim 8, wherein the RF energy is adjusted to provide substantially the same RF energy for each successive proton beam operating cycle.

10. The method according to claim 8, wherein the RF energy is adjusted to provide RF energy during both the on-time and the off-time of the proton beam operating cycle.
